(19) United States
(12) Patent Application Publication
(10) Pub. No.: US 2014/0274811 A1
(43) Pub. Date: Sep. 18, 2014

(54) Methods for Amplifying a Complete Genome or Transcriptome

(71) Applicant: Lyle J. Arnold, Poway, CA (US)

(72) Inventor: Lyle J. Arnold, Poway, CA (US)

(21) Appl. No.: 14/214,634

(22) Filed: Mar. 14, 2014

Related U.S. Application Data
(60) Provisional application No. 61/784,101, filed on Mar. 14, 2013.

(51) Int. Cl. C12N 15/10

(52) U.S. Cl. CPC C12N 15/1065
USPC 506/26

(57) ABSTRACT

The present invention provides methods for amplifying a complete genome or transcriptome. The genome or transcriptome is prepared as a set of target nucleic acids and mixed with a first primer. The first primer comprises a target-binding region having a first random sequence of about 6 to about 9 nucleotides and a tag sequence that contains one or more non-natural nucleotides. The first primer is annealed to the target nucleic acids and extended by polymerase to produce a first duplex nucleic acid. The target nucleic acid is removed from the first nucleic acid. A second primer is annealed to the first nucleic acid having a second random sequence of about 6 to about 9 nucleotides and a tag sequence that contains one or more non-natural nucleotides. The second primer is extended by polymerase to produce a second duplex nucleic acid. The second nucleic acid contains a tag sequence on one terminus and a complement of the tag sequence on the other. The first nucleic acid is removed from the second nucleic acid. A third primer is annealed to the second nucleic acid having the same sequence as the tag sequence or a portion thereof and at least one of the non-natural nucleotides of the tag sequence. The third primer is extended by polymerase to produce a third duplex nucleic acid. The second nucleic acid is removed from the third nucleic acid. The third primer is annealed to the second nucleic acid and the third nucleic acid. The third primer is extended by polymerase. Repeating these last three steps thereby results in amplification of the genome or transcriptome.

### CROSS-REFERENCE TO RELATED APPLICATIONS

This application is a non-provisional patent application of provisional patent application Ser. No. 61/784,101 filed Mar. 15, 2013 incorporated herein in its entirety.

### STATEMENT REGARDING FEDERALLY SPONSORED RESEARCH OR DEVELOPMENT

### None

### INCORPORATION-BY-REFERENCE OF MATERIAL SUBMITTED ON COMPACT DISC

### None

### BACKGROUND OF THE INVENTION

(1) Field of the Invention

The present invention relates to methods of amplifying a target nucleic acid. Specifically, methods for the amplification of an entire or complete genome or transcriptome.

(2) Description of Related Art

Whole genome amplification is an increasingly common technique through which minute amounts of DNA or RNA may be amplified to generate quantities suitable for genetic testing and analysis. However, current methods known in the art can be slow, tedious, cumbersome and expensive to perform. They also have disadvantages such as amplification-induced errors and template bias. These issues result in sub-optimal performance and less than desirable overall utility for these methods. The present invention describes methods for whole genome and whole transcriptome amplification that overcome these disadvantages.

### BRIEF SUMMARY OF THE INVENTION

The present invention provides methods for amplifying a complete genome or transcriptome for further manipulation.

In one aspect of the present invention, a method is provided for amplifying a complete genome or transcriptome. Genomic DNA (gDNA) may be obtained from a variety of samples, such as blood. The gDNA is prepared for amplification by a variety of methods commonly known in the art such as for example using the QIAamp DNA Blood Mini Kit from QIAGEN (Venlo, Netherlands). Alternatively, messenger RNA (mRNA), may be prepared from a sample using a variety of methods commonly known in the art, such as the Dynabeads™ mRNA Purification kit from Life Technologies (Carlsbad, Calif.). The nucleic acids within these starting materials will consist of a mixture of fragments as a result of the purification process. In addition, they may or may not be intentionally fragmented further to generate a desired distribution of sizes. This fragmented gDNA or mRNA starting material will be referred to as “target nucleic acids”.

The target nucleic acids are mixed with a first primer. The first primer comprises a target-binding region having a first random sequence of about 6 to about 9 nucleotides and a tag sequence that contains one or more non-natural nucleotides. The primer is annealed to the target nucleic acids and extending by polymerase to produce a first duplex nucleic acid for each fragment. Each duplex contains a target nucleic acid and a first nucleic acid.

The target nucleic acid is removed from the first nucleic acid. A second primer is annealed to the first nucleic acid. The second primer comprises a target-binding region having a second random sequence of about 6 to about 9 nucleotides and a tag sequence that contains one or more non-natural nucleotides. The second primer is extended by polymerase to produce a second nucleic acid duplex containing said first nucleic acid and a second nucleic acid. The second nucleic acid contains a tag sequence on one end and a complement of the tag sequence on the other.

The first nucleic acid is removed from the second nucleic acid. A third primer that is the same sequence as the tag sequence or a portion thereof, but specifically including at least one of the non-natural nucleotides contained in the tag sequence, is annealed to the second nucleic acid and extended by polymerase to produce a third nucleic acid duplex. The third nucleic acid duplex contains the second nucleic acid and a third nucleic acid. This third duplex is now amplified for all target nucleic acids by repeating the cycle of removing the strands from one another (e.g., thermal denaturation), annealing the third primer (binds to both strands) and extending the third primer, thus amplifying the whole genome or transcriptome.

The use of at least one non-natural nucleotide (that binds its complementary non-natural nucleotide but not the natural nucleotides) in the second primer results in directed binding to only the tag sequences in the third nucleic acid duplex during amplification. This greatly reduces or eliminates mispriming to either non-target nucleic acids or only a subset of target nucleic acids that often leads to amplification bias. This also decreases primer dimerization.

In one embodiment the first random sequence of the first primer and the second random sequence of the second primer have the same sequence.

In another embodiment of this aspect of the present invention the primer comprises a tag sequence and a target-binding region having a random sequence of about 1 to about 3 nucleotides and a poly-T sequence. Alternatively, the primer may comprise a tag sequence and a target-binding region having a sequence complementary to the target nucleic acid and a poly-T sequence. In addition, the tag sequence may further comprise an anchor.

Other aspects of the invention are found throughout the specification.

### BRIEF DESCRIPTION OF THE DRAWINGS

FIG. 1 is a schematic diagram of one aspect of the present invention utilizing an oligonucleotide with a unique tail sequence for target nucleic acid amplification.

FIG. 2 is a schematic diagram of another aspect of the present invention utilizing the unique tail sequences to capture and purify amplicons produced in FIG. 1.

### DETAILED DESCRIPTION OF THE INVENTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

The term “oligonucleotide” as used herein refers to a polymeric form of nucleotides, either ribonucleotides or deoxyribonucleotides, incorporating natural and non-natural nucleotides of a length ranging from at least 2, or generally about 5 to about 200, or more commonly to about 100. Thus, this term includes double- and single-stranded DNA and RNA. In addition, oligonucleotides may be nuclease resistant and include but are not limited to 2′-O-methyl ribonucleotides, phosphorothioate nucleotides, phosphorodithioate nucleotides, phosphoramidate nucleotides, and methylphosphonate nucleotides.

The term “target,” “target sequence,” or “target nucleic acid” as used herein refers to a nucleic acid that contains a polynucleotide sequence of interest, for which purification, isolation, capture, immobilization, amplification, identification, detection, quantitation, mass determination and/or sequencing, and the like is/are desired. The target sequence may be known or not known, in terms of its actual sequence.

The term “primer” or “primer sequence” as used herein are nucleic acids comprising sequences selected to be substantially complementary to each specific sequence to be amplified. More specifically, primers are sufficiently complementary to hybridize to their respective targets. Therefore, the primer sequence need not reflect the exact sequence of the target. Non-complementary bases or longer sequences can be interspersed into the primer, provided that the primer sequence has sufficient complementarity with the sequence of the target nucleic acid to permit hybridization and extension.

In addition, primers may be nuclease resistant and include primers that have been modified to prevent degradation by exonucleases. In some embodiments, the primers have been modified to protect against 3′ or 5′ exonuclease activity. Such modifications can include but are not limited to 2′-O-methyl ribonucleotide modifications, phosphorothioate backbone modifications, phosphorodithioate backbone modifications, phosphoramidate backbone modifications, methylphosphonate backbone modifications, 3′ terminal phosphate modifications and 3′ alkyl substitutions. In some embodiments, the primer(s) and/or probe(s) employed in an amplification reaction are protected against 3′ and/or 5′ exonuclease activity by one or more modifications.

The skilled artisan is capable of designing and preparing primers that are appropriate for extension of a target sequence. The length of primers for use in the methods and compositions provided herein depends on several factors including the nucleotide sequence identity and the temperature at which these nucleic acids are hybridized or used during in vitro nucleic acid extension. The considerations necessary to determine a preferred length for the primer of a particular sequence identity are well known to the person of ordinary skill.

The term “sample” as used herein refers to essentially any sample containing the desired target nucleic acid(s), including but not limited to tissue or fluid isolated from a human being or an animal, including but not limited to, for example, blood, plasma, serum, spinal fluid, lymph fluid, tears or saliva, urine, semen, stool, sputum, vomit, stomach aspirates, bronchial aspirates, organs, muscle, bone marrow, skin, tumors and/or cells obtained from any part of the organism; plant material, cells, fluid, etc.; an individual bacterium, groups of bacteria and cultures thereof; water; environmental samples, including but not limited to, for example, soil water and air; semi-purified or purified nucleic acids from the sources listed above, for example; nucleic acids that are the result of a process, such as template formation for sequencing, including next generation sequencing, sample processing, nuclease digestion, restriction enzyme digestion, replication, and the like.

The term “amplifying” or “amplification” as used herein refers to the process of creating nucleic acid strands that are identical or complementary to a complete target nucleic acid sequence, or a portion thereof, or a universal sequence that serves as a surrogate for the target nucleic acid sequence. The term “identical” as used herein refers to a nucleic acid having the same or substantially the same nucleotide sequence as another nucleic acid.

The term “nucleic acid” as used herein refers to a polynucleotide compound, which includes oligonucleotides, comprising nucleosides or nucleoside analogs that have nitrogenous heterocyclic bases or base analogs, covalently linked by standard phosphodiester bonds or other linkages. Nucleic acids include RNA, DNA, chimeric DNA-RNA polymers or analogs thereof. In a nucleic acid, the backbone may be made up of a variety of linkages, including one or more of sugar-phosphodiester linkages, peptide-nucleic acid (PNA) linkages (PCT No. WO 95/32305), phosphorothioate linkages, methylphosphonate linkages, or combinations thereof. Sugar moieties in a nucleic acid may be ribose, deoxyribose, or similar compounds with substitutions, e.g., 2′ methoxy and 2′ halide (e.g., 2′-F) substitutions.

Nitrogenous bases may be conventional bases (A, G, C, T, U), non-natural nucleotides such as isocytosine and isoguanine, analogs thereof (e.g., inosine; The Biochemistry of the Nucleic Acids 5-36, Adams et al., ed., 11th ed., 1992), derivatives of purine or pyrimidine bases (e.g., N⁴-methyl deoxyguanosine, deaza- or aza-purines, deaza- or aza-pyrimidines, pyrimidines or purines with altered or replacement substituent groups at any of a variety of chemical positions, e.g., 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines, or pyrazolo-compounds, such as unsubstituted or 3-substituted pyrazolo[3,4-d]pyrimidine (e.g. U.S. Pat. Nos. 5,378,825, 6,949,367 and PCT No. WO 93/13121).

Nucleic acids may include “abasic” positions in which the backbone does not have a nitrogenous base at one or more locations (U.S. Pat. No. 5,585,481), e.g., one or more abasic positions may form a linker region that joins separate oligonucleotide sequences together. A nucleic acid may comprise only conventional sugars, bases, and linkages as found in conventional RNA and DNA, or may include conventional components and substitutions (e.g., conventional bases linked by a 2′ methoxy backbone, or a polymer containing a mixture of conventional bases and one or more analogs). The term includes “locked nucleic acids” (LNA), which contain one or more LNA nucleotide monomers with a bicyclic furanose unit locked in a RNA mimicking sugar conformation, which enhances hybridization affinity for complementary sequences in ssRNA, ssDNA, or dsDNA (Vester et al., 2004, Biochemistry 43(42):13233-41).

The term “releasing” or “released” as used herein refers to separating the desired amplified nucleic acid from its template by heating the duplex to a temperature that denatures the nucleic acid duplex forming two separate oligonucleotide strands.

The term “removing” as used herein refers to a variety of methods used to isolate or otherwise remove and separate one nucleic acid strand of a duplex from another, such as for example enzymatic, thermal and/or chemical digestion, degradation and/or cleavage of one of the strands of the duplex, or denaturation/dissociation of the strands by heat, acoustic energy, chemicals, enzymes or a combination thereof.

The terms “tag region” or “tag sequence” refer to a user-defined nucleic acid sequence or sequences that are incorporated into an oligonucleotide or other nucleic acid structure, such as a primer, to provide one or more desired functionalities. Examples of such elements include, for example, adapters, sequencing primers, amplification primers, capture and/or anchor elements, hybridization sites, promoter elements, restriction endonuclease site, detection elements, mass tags, barcodes, binding elements, and/or non-natural nucleotides. Other elements include those that clearly differentiate and/or identify one or more nucleic acids or nucleic acid fragments in which a tag sequence has been incorporated from other nucleic acids or nucleic acid fragments in a mixture, elements that are unique in a mixture of nucleic acids so as to minimize cross reactivity and the like and elements to aid in the determination of sequence orientation. Some or all of the elements in a tag sequence can be incorporated into amplification products.

The term “hybridization,” “hybridize,” “anneal” or “annealing” as used herein refers to the ability, under the appropriate conditions, for nucleic acids having substantial complementary sequences to bind to one another by Watson & Crick base pairing. Nucleic acid annealing or hybridization techniques are well known in the art. See, e.g., Sambrook, et al., Molecular Cloning: A Laboratory Manual, Second Edition, Cold Spring Harbor Press, Plainview, N.Y. (1989); Ausubel, F. M., et al., Current Protocols in Molecular Biology, John Wiley & Sons, Secaucus, N.J. (1994). The term “substantial complementary” as used herein refers both to complete complementarity of binding nucleic acids, in some cases referred to as an identical sequence, as well as complementarity sufficient to achieve the desired binding of nucleic acids. Correspondingly, the term “complementary hybrids” encompasses substantially complementary hybrids.

The term “anchor sequence” or “anchor” as used herein refers to a user-defined sequence that is added onto a nucleic acid target sequence, often by incorporation via a tag sequence. The anchor may be used to facilitate subsequent processing, such as sequencing, for example, to purify, capture, immobilize or otherwise isolate the target nucleic acid bearing the anchor.

General methods for amplifying nucleic acid sequences have been well described and are well known in the art. Any such methods can be employed with the methods of the present invention. In some embodiments, the amplification uses digital PCR methods, such as those described, for example, in Vogelstein and Kinzler (“Digital PCR,” PNAS, 96:9236-9241 (1999); incorporated by reference herein in its entirety). Such methods include diluting the sample containing the target region prior to amplification of the target region. Dilution can include dilution into conventional plates, multiwell plates, nanowells, as well as dilution onto micropads or as microdroplets. (See, e.g., Beer N R, et al., “On-chip, real time, single copy polymerase chain reaction in picoliter droplets,” Anal. Chem. 79(22):8471-8475 (2007); Vogelstein and Kinzler, “Digital PCR,” PNAS, 96:9236-9241 (1999); and Pohl and Shih, “Principle and applications of digital PCR,” Expert Review of Molecular Diagnostics, 4(1):41-47 (2004); all of which are incorporated by reference herein in their entirety.) In some embodiments, the amplification is by digital PCR.

In some cases, the enzymes employed with the methods of the present invention for amplification of the target region include but are not limited to high-fidelity DNA polymerases, for example DNA polymerases that have 3′-5′ exonuclease proof-reading capabilities. Examples of enzymes that can be used with the methods include but are not limited to AmpliTaq, Phusion HS II, Deep Vent, and Kapa HiFi DNA polymerase.

High-fidelity enzymes allow for high-fidelity (highly accurate) amplification of a target sequence. In some embodiments, the enzymes employed will include high-fidelity DNA polymerases, for example DNA polymerases that have 3′-5′ exonuclease proofreading capabilities. Enzymes that can be used with the methods include but are not limited to AmpliTaq, Phusion HS II, Deep Vent, and Kapa HiFi DNA polymerase.

The amplification product can be detected/analyzed using a number of methods known to those skilled in the art including, but not limited to, fluorescence, electrochemical detection, gel analysis and sequencing. Furthermore, the product can be quantitated using a number of methods known to those skilled in the art such as real time amplification. Quantitation can be normalized by comparison to so-called “house-keeping genes” such as actin or GAPDH or to an internal control that can be added to the reaction in a known amount. Such methods are well known and have been described in Sambrook and Russell, Molecular Cloning: A Laboratory Manual (3rd Ed.) (2001).

Instrumentation for performing the methods described herein is readily available. Such instruments can include instruments for real-time and end-point PCR assays, emulsion PCR, solid-phase PCR, melting curve analyses, and sequencing analyses. Such instruments include Life Technologies 7500 Fast Dx real-time instrument (which is also capable of high-resolution melting curve analyses) and the 3500×1 capillary gel instruments. Other instruments known in the art to be useful in the methods of the present invention are also contemplated for use by one of skill in the art in practicing the methods of the present invention.

The present invention provides methods for amplifying a complete genome or transcriptome.

In one aspect of the present invention, a method of amplifying a complete genome or transcriptome obtained from a sample is described. As starting material, the method of the present invention utilizes genomic DNA (gDNA) or messenger RNA (mRNA) which are prepared from a sample using any of a number of methods commonly known in the art. The nucleic acids within these starting materials will consist of a mixture of fragments as a result of the purification process, and may or may not be intentionally fragmented further to generate a distribution of fragments of the desired size.

These target nucleic acids are mixed with a first primer. The primer comprises a random target-binding region having about 6 to about 9 nucleotides and a tag sequence that contains one or more non-natural nucleotides. The primer is annealed to the target nucleic acids and extending by polymerase to produce a first duplex nucleic acid for each fragment. Each duplex contains the target nucleic acid and a first nucleic acid.

The target nucleic acid is removed from the first nucleic acid. The first primer is annealed to the first nucleic acid and extending by polymerase to produce a second nucleic acid duplex containing said first nucleic acid and a second nucleic acid. The second nucleic acid contains a tag sequence on one end and a complement of the tag sequence on the other.

The first nucleic acid is removed from the second nucleic acid. A second primer that is the same sequence as the tag sequence or a portion thereof, but specifically including at least one of the non-natural nucleotides contained in the tag sequence, is annealed to the second nucleic acid and extended by polymerase to produce a third nucleic acid duplex. The third nucleic acid duplex contains the second nucleic acid and a third nucleic acid.

This third duplex is amplified for all target nucleic acids by repeating the cycle of removing the strands from one another (e.g., thermal denaturation), annealing the second primer to both strands and extending the second primer, thus amplifying the whole genome or transcriptome.

In a first embodiment, a single primer or set of primers may be used for binding the target nucleic acids. Each primer comprises a random target binding region, preferably about 6-9 nucleotides in length, and a tag sequence containing unique non-natural nucleotides, such as L-ribose or isoC and isoG. The non-natural nucleotides will bind to the complementary non-natural nucleotides but are not capable of binding natural nucleic acids (i.e., A, C, G, T and U). In a preferred embodiment, a single primer is used for binding the target nucleic acids.

In another embodiment, 2 or more primers may be used. In this case, the primers may all share the same target binding sequence (e.g., a random sequence 6 to 9 nucleotides in length) but have different tag regions to introduce distinct functionality on each side of the produced nucleic acid. For example, the produced nucleic acid may have distinct primer binding sites on the two termini and/or a barcode sequence on one terminus and not the other.

The utility of introducing complementary tail sequences within the produced nucleic acid through the primer provides a method of selecting sequences of a relatively consistent size for amplification. Initially a collection of random length “tailed” species will be generated. Of these generated species, the shorter sequences will not dominate the amplification reaction because their tail sequences preferentially bind to each other rather than the primer. Consequently, longer species will be favored in the amplification reaction.

In a similar way species that are exceptionally long will often not extend sufficiently to incorporate tails at both their termini. Consequently, subsequent exponential amplification will not be possible. Therefore, a mid-range of species from between about 100 to about 500 base pairs will be generated across the entire set of target nucleic acids.

In a second embodiment, mRNA is the desired target nucleic acid. In this method, the mRNA may be converted into a first cDNA strand by using a primer that targets the poly-A junction region or the poly-A tail. A generic primer containing a target binding region that comprises a short random nucleic acid sequence of 1 to about 3 base pairs in length, referred to as the wobble sequence, together with a poly-T region containing about 8 to about 15 nucleotides in length may be used to prime the junction region. A primer that comprises a poly-T region containing about 8 to about 18 nucleotides in length may also be used. The chosen primer is annealed to the target mRNAs and extended by polymerase. The mRNA strands are then removed (e.g. by RNaseH) and the cDNAs produced may then be amplified with the methods of the present invention.

Alternatively, the primers may further comprise a tag sequence such as the T1 tag shown in FIG. 1. After primer extension and removal of the RNA target nucleic acid (e.g. by RNaseH), the first nucleic acid produced comprises the T1 tag.

It is also anticipated that the primer sequences used for DNA or RNA target nucleic acids may be specific, as opposed to random primers.

In a third embodiment, applications of these methods may be utilized for further manipulation of the amplicons including for example detection and/or sequencing. More specifically, the unique engrafted tail sequences from the amplification method above may be used to simplify a broad range of subsequent manipulations. For example, the unique tail sequences may be utilized to capture and purify the amplicon products. Providing complementary sequences to the tail sequences on a solid support, such as a magnetic bead, amplicons may be captured and then purified from the reaction mixture by elution. In specific applications it may be beneficial to have a single amplicon bound to a solid support. This can be achieved when the number of magnetic beads, for example, exceeds the number of amplicons in the reaction mixture.

In a second application, the amplicon bound beads may be deposited into single pores or wells, amplified further if desired and sequenced.

In a third application, the unique tail sequences incorporated into the amplicons may be utilized for detection. For example, a probe comprising a sequence that is identical to the tag sequence of the first primer or a portion thereof may be used to bind the amplicon containing the complementary tag sequence for detection. The probe may also be a molecular beacon. In this embodiment, binding results in a conformational change in the probe initiating a fluorescent signal that can be detected.

In a fourth application, the tail sequences may be converted into anchor sequences and transferred to conventional sequencing instruments. Sequences may then go directly into DNA sequencing by synthesis using appropriate anchor constructs.

The information set forth above is provided to give those of ordinary skill in the art a complete disclosure and description of how to make and use the embodiments of the device and methods, and are not intended to limit the scope of what the inventor regards as his invention. Modifications of the above-described modes (for carrying out the invention that are obvious to persons of skill in the art) are intended to be within the scope of the following claims. All publications, patents, and patent applications cited in this specification are incorporated herein by reference. For example, many of the wash steps cited in the different methods are optional as are some of the steps that remove and/or separate two nucleic acid strands from one another. Not performing at least some of the wash and/or separation steps will afford a faster, simpler and more economical work flow, while still achieving the desired results. In another example, the stepwise addition/binding of certain oligonucleotides and/or target nucleic acids in the exemplified methods may be combined. Furthermore, a variety of polymerases, extension conditions and other amplification protocols known to those skilled in the art may be used in various steps or combination of steps in the methods described above. Other obvious modifications to the methods disclosed that would be obvious to those skilled in the art are also encompassed by this invention.

I claim:

1. A method of amplifying a genome or transcriptome obtained from a sample, wherein said genome or transcriptome is provided as a set of target nucleic acids said method comprising the steps of:
A. annealing a first primer to a set of target nucleic acids in a mixture comprising said target nucleic acids and said first primer wherein said primer comprises a target binding region containing a first random sequence having about 6 to about 9 nucleotides and a tag sequence, wherein said tag sequence contains one or more non-natural nucleotides and extending said first primer to produce a first duplex nucleic acid containing a first nucleic acid and said target nucleic acid, and optionally removing said first nucleic acid from said target nucleic acid;
B. annealing a second primer to said first nucleic acid, wherein said second primer comprises a target binding region containing a second random sequence having about 6 to about 9 nucleotides and a tag sequence, wherein said tag sequence contains one or more non-natural nucleotides and extending said second primer by polymerase to produce a second nucleic acid duplex containing said first nucleic acid and a second nucleic acid containing a tag sequence on one end and a complement of said tag sequence on the other and, optionally removing said first nucleic acid from said second nucleic acid;
C. annealing a third primer having a substantially identical sequence as said tag sequence or a portion thereof and at least one of said non-natural nucleotides of said tag sequence to said second nucleic acid and extending said third primer by polymerase to produce a third nucleic acid duplex containing said second nucleic acid and a third nucleic acid and, optionally removing said second nucleic acid from said third nucleic acid;
D. annealing said third primer to said second nucleic acid and said third nucleic acid and extending said third primer bound to said second nucleic acid and said third nucleic acid by polymerase; and
E. repeating steps F and G thereby amplifying said genome or said transcriptome.

2. The method according to claim 1, wherein said first random sequence of said first primer and said second random sequence of said second primer are the same.

3. The method according to claim 1, wherein said first primer comprises a tag sequence and a target-binding region having a random sequence of about 1 to about 3 nucleotides and a poly-T sequence.

4. The method according to claim 1, wherein said first primer comprises a tag sequence and a target-binding region comprising a poly-T sequence.

5. The method according to claim 1, wherein said first primer further comprises an anchor sequence.
